(19) United States
(12) Patent Application Publication
(10) Pub. No.: US 2017/0303767 A1
(43) Pub. Date: Oct. 26, 2017

(54) ENDOSCOPE FOR STORING IMAGES

(71) Applicant: HOYA CORPORATION, Tokyo (JP)

(72) Inventors: Masaaki FUKUDA, Tokyo (JP); Junko ISAWA, Tokyo (JP); Akihiro ITO, Saitama (JP)

(73) Assignee: HOYA CORPORATION, Tokyo (JP)

(21) Appl. No.: 15/644,314

(22) Filed: Jul. 7, 2017

Related U.S. Application Data
(63) Continuation of application No. 13/951,844, filed on Jul. 26, 2013.

(30) Foreign Application Priority Data
Aug. 10, 2012 (JP) 2012-178421

(51) Int. Cl. A61B 1/00

(52) U.S. Cl. CPC A61B 1/0002; A61B 1/0005

(57) ABSTRACT

An endoscope is provided having a video compiler and a recorder. The video compiler continuously receives image signals and creates a video file with the image signals. The recorder receives and stores the video file. The video compiler writes a flag into the video file when a first event occurs.

### CROSS-REFERENCE TO RELATED APPLICATIONS

This is a continuation application of pending U.S. patent application Ser. No. 13/951,844, filed on Jul. 26, 2013, which claims priority to JAPAN Application No. 2012-178421, filed Aug. 10, 2012. The disclosures of these documents, including the specifications, drawings and claims, are incorporated herein by reference in their entirety.

### BACKGROUND OF THE INVENTION

### Field of the Invention

The present invention relates to an imager for storing an image.

### SUMMARY OF THE INVENTION

An object of the present invention is to provide an endoscope for storing a series of object images as a video, and cataloguing the location of an arbitrary still image in the video.

According to the present invention, an endoscope is provided that comprises a video compiler and a recorder. The video compiler continuously receives image signals and creates a video file with the image signals. The recorder receives and stores the video file. The video compiler writes a flag into the video file when a first event occurs.

### BRIEF DESCRIPTION OF THE DRAWINGS

The objects and advantages of the present invention will be better understood from the following description, with reference to the accompanying drawings in which:

FIG. 1 is a block diagram showing the endoscope as an embodiment of the present invention;

FIG. 2 is a block diagram showing the header of a transport packet that is comprised in a video file;

FIG. 3 is a block diagram showing a video-recording controller;

FIG. 4 is a block diagram showing a video-retrieving controller;

FIG. 5 is a flowchart showing a video-recording process;

FIG. 6 is a flowchart showing a recording-initialization process;

FIG. 7 is a flowchart showing a recording process;

FIG. 8 is a flowchart showing an event process;

FIG. 9 is a flowchart showing a video-playing process;

FIG. 10 is a monitor that displays search results; and

FIG. 11 is a monitor that displays search results.

### DESCRIPTION OF THE PREFERRED EMBODIMENTS

The present invention is described below with reference to the embodiments shown in the drawings.

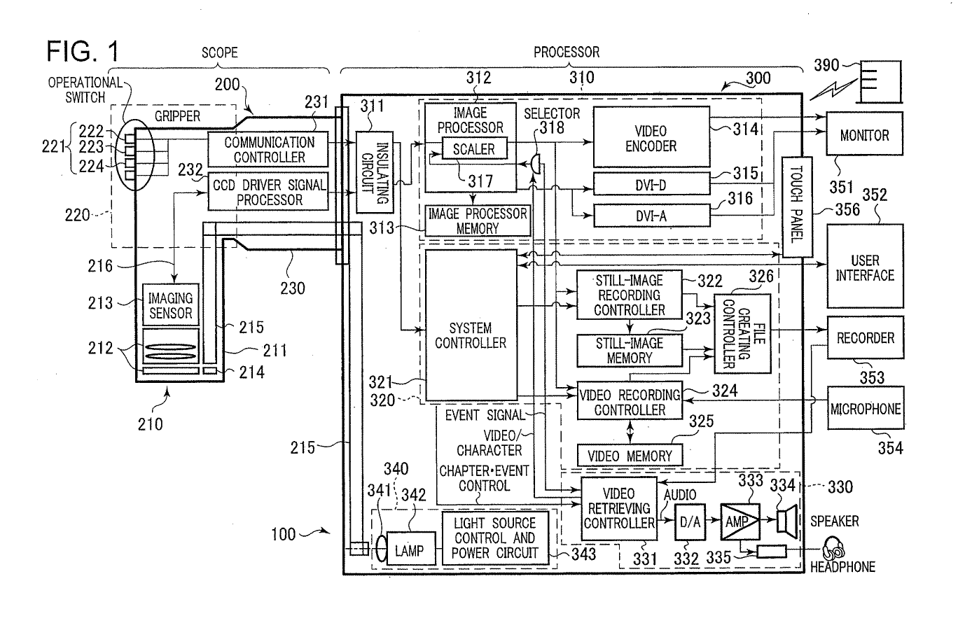

The constructions of an endoscope 100 are described hereinafter with reference to FIG. 1.

The endoscope 100 mainly comprises a scope 200, a processor 300, a server 390, a monitor 351, and a recorder 353 that is a USB memory device or hard disk, for example.

The scope 200 comprises an insertion part 210 that is inserted inside of an observational object, e.g., a human body, a gripper 220 that is held by a user, and a connector 230. The gripper 220 is connected to the processor 300 by the connector 230.

A flexible tube 210 is provided at the tip of the insertion part 210. A photographic lens 212, an imaging sensor 213, and an illumination fiber 215 are provided in the flexible tube 210.

The illumination fiber 215 emits light created by a light-source block 340, and illuminates the object through the illumination lens 214.

The photographic lens 212 projects an object image onto the imaging sensor 213. The imaging sensor 213 photographs the object image and outputs an image signal. The image signal is sent to the connector 230 through an image signal wire 216 that is provided in the flexible tube 211.

The gripper 220 comprises a plurality of operational switches 221 that are projected from a surface of the gripper 220. The operational switch 221 comprises a still-image recording switch 222, a freeze switch 223, and a video-recording switch 224. When the still-image recording switch 222 is depressed, a still image is stored in the recorder 353. When the freeze switch 223 is depressed, a video displayed on the monitor 351 is temporarily stopped. When the recording switch 224 is depressed, recording of a video is started or stopped. Therefore, a user operates the operational switch 221 so as to control the operation of the endoscope 100.

The connector 230 comprises a communication controller 231 and a processing circuit 232. The communication controller 231 receives a signal from the operational switch 221 and sends the signal to the processor 300. The processing circuit 232 comprises a CCD driver and a signal processor. The CCD driver sends a driving signal to the imaging sensor 213. The signal processor receives an image signal from the imaging sensor 213, processes the received image signal, and sends the processed image signal to the processor 300.

The processor 300 is described below. The processor 300 comprises an insulating circuit 311, an image-processing block 310, a control block 320, a retrieving block 330, and the light source block 340. The insulating circuit 311 insulates the scope and the processor 300. The image-processing block 310 processes an image that is received from the scope 200. The control block 320 controls the endoscope 100. The retrieving block 330 retrieves an image from the recorder 353. The light source block 340 creates illumination light.

The image-processing block 310 is described below. The image-processing block 310 mainly comprises an image processor 312, an image processor memory 313, a display video encoder 314, a DVI-D converter 315, a DVI-A converter 316, and a selector 318.

While a user observes an object, the image processor 312 receives image signals continuously from the imaging sensor 213 through the insulating circuit 311, and creates frame images continuously. The image processor 312 uses the image processor memory 313 as a temporary memory and processes the received image signals continuously so that the frame images are created. During this process, the scaler 317 processes image signals to properly adjust their aspect ratios, vertical frequency, and quality of frame image. The created frame is sent to the display video encoder 314, a still-image storing controller 322 and a video storing controller 324 that are described hereinafter. A user can recognize frame images that are continuously displayed on the monitor 351 as video.

While a stored video is played, the image processor 312 receives image signals and event signals through the selector 318 from the video-retrieving controller 331, and creates frame images continuously using the image processor memory 313 as a temporary memory. During this process, the scaler 317 processes image signals to properly adjust their aspect ratios, vertical frequency and quality of frame image. The created frame images are sent to the display video encoder 314. The video-retrieving controller 331 and the event signal are described hereinafter.

The display video encoder 314 converts the frame images to a format that can be displayed by the monitor 351, and outputs to the monitor 351. The DVI-D converter 315 converts the format of the frame images to DVI-D format, and outputs to the monitor 351. The DVI-A converter 316 converts the format of the frame images to the DVI-A format, and outputs to the monitor 351.

The control block 320 is described below. The control block 320 comprises a system control circuit 321, a still-image recording controller 322, a still-image memory 323, a video storing controller 324, a video memory 325, and a file creating controller 326.

The system control circuit 321 is connected to a user interface 352 and a touch panel 356, which may be a keyboard, mouse, footswitch or menu button, and receives instructions from a user through the user interface 352 and the touch panel 356. Then, it controls the endoscope according to the received instructions.

The system control circuit 321 determines what event occurred when the user interface 352 or the touch panel 356 is operated, creates an event register, and sends the event register as the event signal to the video-recording controller 324. The event signal is an electric signal containing the event register. The event register is expressed with six digits in hexadecimal notation, and comprises the event ID that is expressed with two digits in hexadecimal notation and the event number that is expressed with four digits in hexadecimal notation. The event ID is determined preliminarily for each type of event. For example, an event ID is “20” in the case that the still-image recording switch is operated, and an event ID is “21” in the case that the freeze switch 223 is operated. The event number is increased by increments of one from zero when an event occurs, and is assigned to each type of event. Therefore, when the still-image recording switch 222 is operated three times, the event ID is “20”, the event number is “0002”, and the event register is “200002”.

The system control circuit 321 crates a chapter register at the start of recording of a video, at the passage of each time interval from the begging of recording the video, and at the end of recording the video. The system control circuit 321 then sends the chapter registers to the video-recording controller 324 as event signals. The event signal is an electric signal containing the chapter register. The chapter register is expressed with six digits in hexadecimal notation, and comprises the chapter ID that is expressed with two digits in hexadecimal notation and the chapter number that is expressed with four digits in hexadecimal notation. The chapter ID is preliminarily determined to be “10”. The chapter number is determined to be “0000” when storing a video begins, and is increased by an increment of one when each time interval passes. Therefore, the chapter register is “100000” when storing video is started. In the case that the time interval is three minutes, the chapter number is “0003” and the chapter register is “100003” when nine minutes have elapsed since the beginning of recording of a video.

The system control circuit 321 creates observation information, and stores it to the recorder 353. The observation information is a table that correlates the event ID, chapter ID, occurrence time of an event or chapter, chapter number, name and occurrence time of the event, information of the patient, information of the observer, and time and date of their observation together. The system control circuit 321 also has a search function. The search function is described hereinafter.

The still-image recording controller 322 receives a frame image from the image processor 312, and creates a still image from the frame image using the still-image memory 323 as a temporary memory. The successive process is still-image capturing. The process of creating still images is described below. When a user depresses the still-image recording switch 222, a signal is sent from the still-image recording switch 222 to the system control circuit 321 through the communication controller 231. The system control circuit 321 that received the signal sends an instruction signal that represents the creation of a still image to the still-image recording controller 322. The still-image recording controller 322 uses a predetermined image compression method to compress a frame image that it received from the image processor 312 when the still-image recording controller 322 receives the signal, and outputs the compressed frame image as a still image. The still image is sent to the file creating controller 326. The frame image is compressed by the JPEG method, for example.

The video-recording controller 324 receives a frame image from the image processor 312, and also an event signal from the system control circuit 321, and creates a video from the frame image using the video memory 325 as a temporary memory. The process of creating video is described below. When a user depresses the recording switch 224, a signal is sent from the still-image recording switch 222 to the system control circuit 321 through the communication controller 231. The system control circuit 321 that received the signal sends an event signal and instruction signal that represents the start of video recording to the video-recording controller 324. The sent event signal has the chapter register “100000” that represents the start of video recording. The video-recording controller 324 uses a predetermined image compression method to compress a frame image that is received from the image processor 312 when the video-recording controller 324 receives the signal that represents the start of video recording, and outputs the chapter register with the compressed frame image as a video. The system control circuit 321 creates a chapter register for each time interval that passes while the recording switch 224 is depressed, that is, since the beginning of recording the video, and sends the chapter register as an event signal to the video-recording controller 324. The video-recording controller 324 uses a predetermined image compression method to compress a frame image that is received from the image processor 312, and then outputs the chapter register with the compressed frame image as a video. In the case that the recording switch 224 is depressed while the video is being recorded, then recording the video is stopped and the video-recording controller 324 outputs the frame image and the chapter register as a video. When a user depresses the user interface 352, a signal is sent from the user interface 352 to the system control circuit 321 through the communication controller 231. The system control circuit 321 that received the signal sends an event signal that corresponds to an operation of the user interface 352 to the video-recording controller 324. The corresponding event signal has an event register. The video-recording controller 324 uses a predetermined image compression method to compress a frame image that is received from the image processor 312 when the video-recording controller 324 receives the event signal, and outputs the event register with the compressed frame image as a video.

The video-recording controller 324 is connected to a microphone 354. When a user speaks into the microphone 354, the microphone 354 converts the user's voice to an audio signal and sends it to the video-recording controller 324. In the case that the video-recording controller 324 is recording a video at that time, the video-recording controller 324 combines and compresses the audio signal and the frame image as a video, and sends the video to the file creating controller 326. The frame image and the audio signal are compressed by MPEG-2 or MPEG-4, for example.

The file creating controller 326 sends the received still image and the received video to the recorder 353 with proper communication protocol. The communication protocol is USB or IEEE1384, for example. The file creating controller 326 may send the received still image and the received video to the server 390 over a wireless LAN. The server 390 receives and records the still image and the video.

The retrieving block 330 is described below. The retrieving block 330 mainly comprises the video-retrieving controller 331, a D/A converter 332, an amplifier 333, a speaker 334, and a headphone jack 335.

The video-retrieving controller 331 is connected to the recorder 353 and the D/A converter 332, and retrieves a video file stored in the recorder 353. Then, it decompresses the video file and produces a frame image, sound signal, and event signal in succession. The frame image and event signal is sent to the image processor 312. The sound signal is sent to the D/A converter 332. The D/A converter 332 converts the sound signal to an analog sound signal and sent it to the amplifier 333. The amplifier 333 amplifies the analog sound signal to an appropriate level, and sends it to the speaker 334 and the headphone jack 335.

The light-source block 340 is described below. The light-source block 340 mainly comprises a condenser lens 341, a lamp 342, and a light-source control and power circuit 343. The light-source control and power circuit 343 is powered from a processor power circuit (not shown) included in the processor 300, and outputs voltage that is appropriate for the lamp 342 to the lamp 342. The lamp 342 receives electric power from the light-source control and power circuit 343 and emits illumination light. The condenser lens 341 focuses the illumination light created by the lamp 342 on the illumination fiber 215, so that the illumination light is provided to the illumination fiber 15.

The function of the system control circuit 321 as a searcher is described below. When a user enters chapter, event, or patient information into the user interface 352 or the touch panel 356 to search for a particular patient, they are transmitted to the system control circuit 321. The information entered by a user is search information. The search information includes chapter number, name and occurrence time of event, information of patient, information of observer, and time and date of observation, etc. The name of an event corresponds to a user's operation of the user interface 352 and the touch panel 356, for example, the operation of the still-image recording switch is assigned to “still-image capturing”, and the operation of the freeze switch 223 is assigned to “video freezing”. The system control circuit 321 that received the search information retrieves observation information that corresponds to the search information. Then, it retrieves a video file that corresponds to the retrieved observation information from the recorder 353. For examples, in the case that a user enters “still image capturing” as search information, the system control circuit 321 retrieves the event ID “20” of observation information that corresponds to “still image capturing”. Then, it retrieves a video file having the event ID “20” from the recorder 353. Alternatively, in the case that a specific video file is designated preliminarily, the system control circuit 321 can retrieve a frame image that corresponds to the retrieved observation information from the video file. For example, in the case that a user enters “still image capturing” as search information, the system control circuit 321 retrieves the event ID “20” of observation information that corresponds to “still image capturing”. Then, it retrieves a frame image having the event ID “20” from the video file. These processes can also be processed for a chapter index.

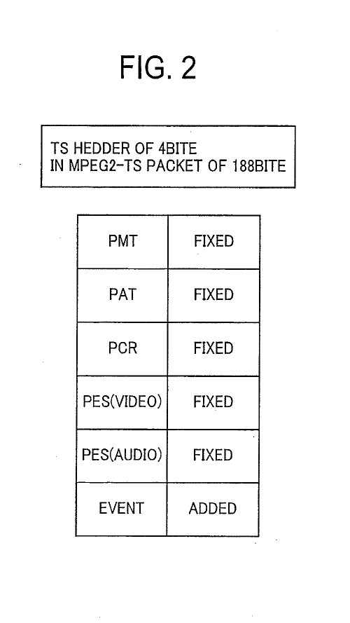

The process for recording an event register is described below with the MPEG-2 compression method with reference to FIG. 2. The transport stream of MPEG-2 divides video data into transport packets and sends them. The transport packet has a packet identifier that records the event register. FIG. 2 shows the construction of the packet identifier of the transport packet. The packet identifier mainly records PMT, PAT, PCR, PES(Video), PES(Audio), and the Event. The PMT, PAT, PCR, PES(Video), and PES(Audio) are common, so that the descriptions of them are omitted. The Event is recorded in a field that can be used freely by a user, and it records the event register.

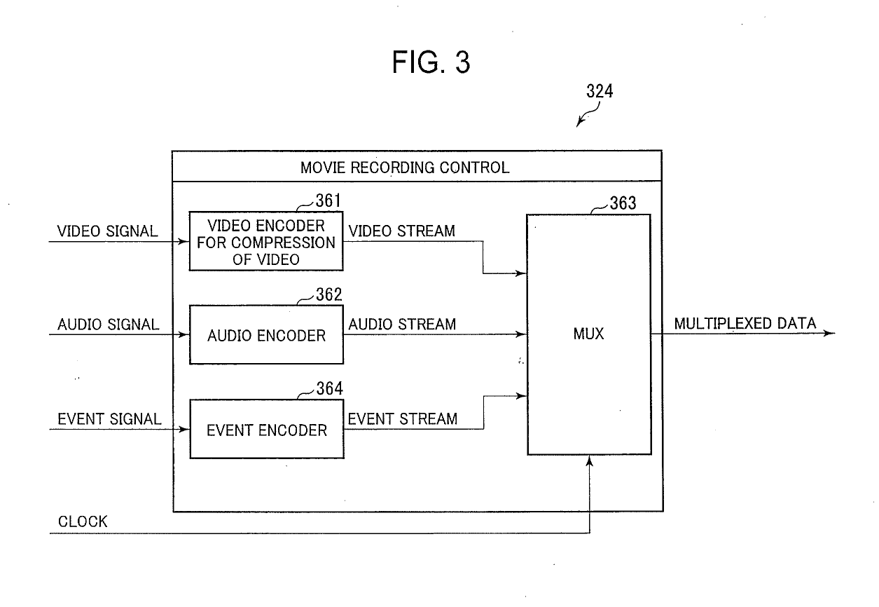

The construction of the video-recording controller 324 is described below with reference to FIG. 3. The video-recording controller 324 mainly comprises a compression video encoder 361, an audio encoder 362, a multiplexer 363, and an event encoder 364. The audio encoder 362 receives a sound signal from the microphone 354, compresses the sound signal, and outputs an audio stream. Therefore, the audio stream is created by compressing a sound signal and then sent to the multiplexer 363. The event encoder 364 receives the event signal from the system control circuit 321, transforms it to an event stream, and outputs the event stream to the multiplexer 363. The compression video encoder 361 receives a frame image from the image processor 312, compresses the frame image to a video stream, and outputs the video stream. Therefore, the video stream is created by compressing frame images of a video and then sent to the multiplexer 363. The multiplexer 363 receives the audio stream, video stream, and event stream, combines and compresses them, and outputs as video.

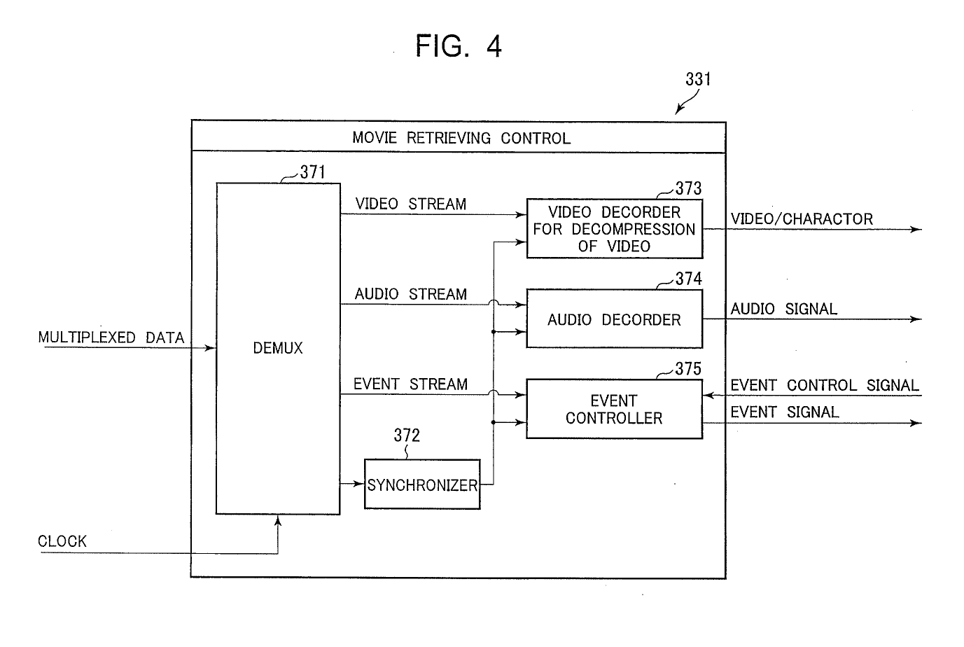

The construction of the video-retrieving controller 331 is described below with reference to FIG. 4. The video-retrieving controller 331 mainly comprises a demultiplexer 371, a synchronizer 372, a video decoder 373, an audio decoder 374, and an event controller 375.

The demultiplexer 371 receives a video file from the recorder 353 and clock signal from a clock generator (not shown). Then, it synchronizes with the clock signal and produces video stream, audio stream, event stream, and a synchronization signal from the video file. The video stream is sent to the video decoder 373. The audio stream is sent to the audio decoder 374. The event stream is sent to the event controller 375. The synchronization signal is sent to the video decoder 373, the audio decoder 374, and the event controller 375 through the synchronizer 372.

The video decoder 373 converts the video stream to frame images and outputs the frame images synchronously with synchronization signals. The audio decoder 374 converts the audio stream to an audio signal and outputs the audio signal synchronously with a synchronization signal.

The event controller 375 converts the event stream to an event signal and outputs the event signal synchronously with a synchronization signal. The frame images, audio signal and event signal are output synchronously with a synchronization signal, so that the frame images, audio signal and event are reproduced without misalignment. The event controller 375 receives an event control signal from the system control circuit 321. The event control signal includes the search information. The event controller 375 that received the search information retrieves observation information that corresponds to the search information, and sends the retrieved observation information to the system control circuit 321. The system control circuit 321 that received the observation information sends the name of the video file that corresponds to the received observation information to the event controller 375. The event controller 375 that received the name of the video file retrieves the video file from the recorder 353, and sends the retrieved video file to the system control circuit 321. Alternatively, in the case that a specific video file is designated preliminarily, the event controller 375 retrieves observation information that corresponds to the search information from the recorder 353, and sends the retrieved observation information to the system control circuit 321. The system control circuit 321 retrieves a frame image that corresponds to the received observation information from the video file. These processes can also be processed for a chapter index.

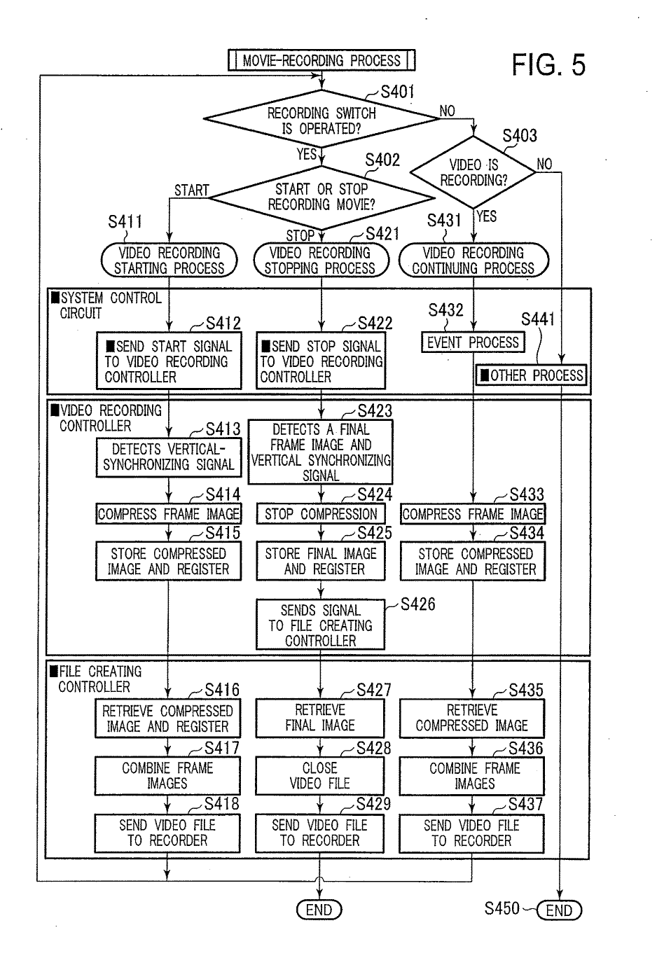

The video-recording process is described below with reference to FIG. 5. The video-recording process is carried out periodically by the processor 300 while the processor 300 is in use.

In Step S401, it is determined whether the recording switch 224 is operated or not. In the case that the recording switch 224 is operated, the process proceeds to Step S402. In the case that the recording switch 224 is not operated the process proceeds to Step S403.

In Step S402, it is determined whether the operation of the recording switch 224 corresponds to the starting or stopping of recording of a video. In the case that it corresponds to the starting of recording of a video, a video is recorded from Step S411 to Step S418. In the case that it corresponds to the stopping of recording of a video, recording of a video is stopped in Step S421 to Step S429.

In Step S403, it is determined whether a video is recording or not. In the case that a video is recording, recording of a video continues from Step S431 to S436. In the case that the video is not recording, another process is carried out and the video recording process ends at Step S450.

The video-recording starting process illustrated from Step S411 to Step S418 is described below. In Step S412, the system control circuit 321 sends a signal that indicates the start of recording to the video-recording controller 324. Step S413 to Step S415 is carried out by the video-recording controller 324. In Step S413, the video-recording controller 324 detects a vertical synchronizing signal from the frame image that is received from the image processor 312. In Step S414, a frame image is compressed. In Step S415, the video memory 325 stores the compressed frame image and event register or chapter register that corresponds to the compressed frame image. The next Steps S416 to S418 are carried out by the file creating controller 326. In Step S416, the file creating controller 326 retrieves the compressed frame image from the video memory 325 through the video-recording controller 324. In Step S417, a plurality of frame images is combined so that a video file is created. In Step S418, the video file is sent and stored in the recorder 353, and the process returns to Step S401.

The video recording stopping process from Step S421 to Step S429 is described below. In Step S422, the system control circuit 321 sends a signal to the video-recording controller 324 indicating that recording of the video is to stop. Steps S423 to S426 are carried out by the video-recording controller 324. In Step S423, the video-recording controller 324 detects a final frame image and vertical synchronizing signal from the frame image that is received from the image processor 312. In Step S424, the final frame image is compressed, and image compression ends. In the next Step S425, the video memory 325 stores the compressed final frame image and event register or chapter register that corresponds to the compressed frame image. In Step S426, the video-recording controller 324 sends a signal that indicates completion of compression of the final frame image to the file creating controller 326. The next Steps S427 to S429 are carried out by the file creating controller 326. In Step S427, the file creating controller 326 retrieves the compressed final frame image from the video memory 325 through the video-recording controller 324. In Step S428, a plurality of frame images is combined so that a video file is created, and then, the video file is closed. In Step S429, the video file is sent to and stored in the recorder 353, and the process ends.

The video-recording continuing process from Step S431 to Step S437 is described below. Step S432 is carried out by the system control circuit 321. In Step S432, an event process, as described hereinafter, is carried out. Steps S433 and S434 are carried out by the video-recording controller 324. In Step S433, the received frame image is compressed. In the next Step S434, the video memory 325 stores the compressed frame image and the event register or chapter register that corresponds to the compressed frame image. The next Steps S435 to S437 are carried out by the file creating controller 326. In Step S435, the file creating controller 326 retrieves the compressed frame image from the video memory 325 through the video-recording controller 324. In Step S436, a plurality of frame images is combined so that a video file is created. In Step S437, the video file is sent to and stored in the recorder 353, and the process returns to Step S401.

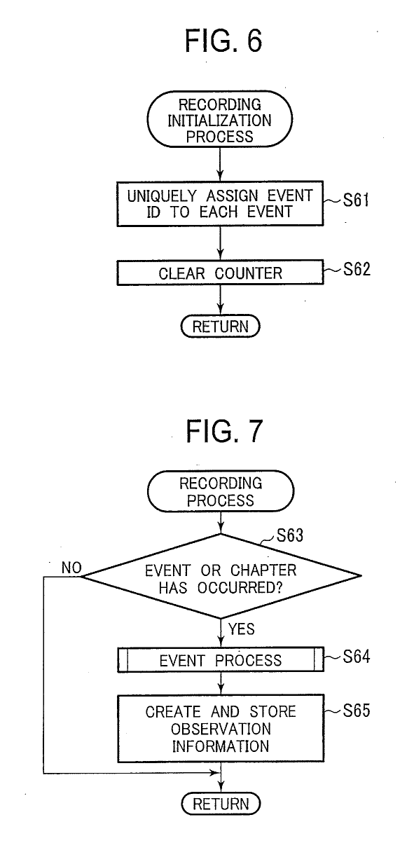

The recording initialization process is described below with reference to FIG. 6. The recording initialization process is undertaken as a part of Step S414 of the video recording process. Therefore, the video-recording controller 324 conducts the recording initialization process in addition to the compression of a frame image.

In Step S61, the video-recording controller 324 assigns a unique event ID to each event conducted by the processor 300. Therefore, the event “still image capturing” is assigned the event ID “20”.

In the next Step S62, the value “0” is substituted for the event counter and chapter counter so as to reset them. Then, the process ends.

The recording process is described below with reference to FIG. 7. The recording process is undertaken as a part of Step S432 of the video recording process. Therefore, the video-recording controller 324 conducts the recording process in addition to the compression of a frame image.

In Step S63, it is determined whether or not an event or chapter has occurred. An event or chapter is determined to have occurred in the case that the system control circuit 321 determines that the user interface 352 or the touch panel 356 has been operated, or that the recording of a video has started, or that each time interval since the beginning of the recording of a video has elapsed, or that recording of a video ends. In the case that an event or chapter has occurred, the process proceeds to Step S64, otherwise, the process ends.

In Step S64, an event process, as described hereinafter, is carried out. The event process creates an event register and chapter register.

The next Step S65, the system control circuit 321 creates observation information, and the recorder 353 stores the observation information. As described hereinbefore, the observation information is a table that correlates the event ID, the chapter ID, occurrence time of an event or chapter, the chapter number, name and occurrence time of the event, patient information, information of observer, and time and date of the observation with each other. Then, the process ends.

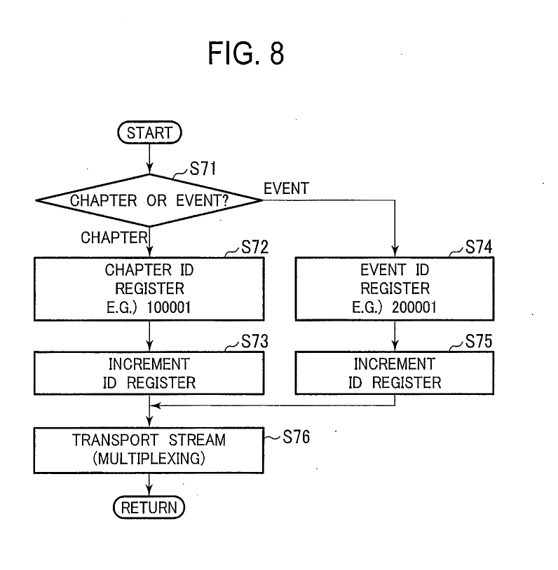

The event process is described below with reference to FIG. 8. The event process is undertaken in Step S65 of the recording process.

In Step S71, it is determined which event or chapter has occurred. In the case that a chapter has occurred, the process proceeds to Step S72. In the case that an event has occurred, the process proceeds to Step S74.

In Step S72, a chapter register is created by the process described hereinbefore. In the next Step S73, the chapter number is increased by one.

In Step S74, an event register is created by the process described hereinbefore. In the next Step S75, the event number that corresponds to the presently occurring event is increased by one.

In the next Step S76, the event and chapter registers which have formats conforming to the transport stream, are output. Then, process ends.

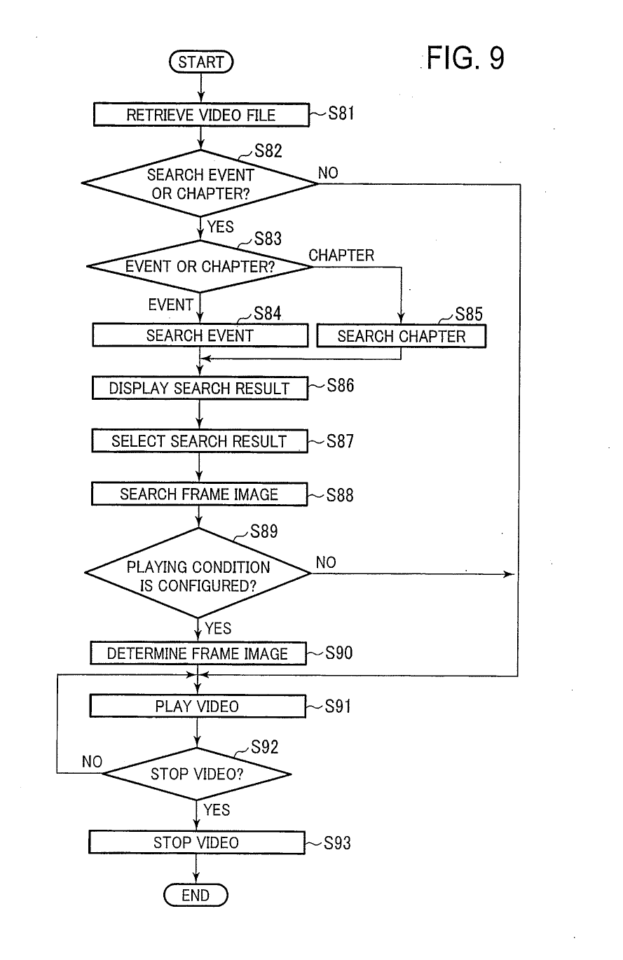

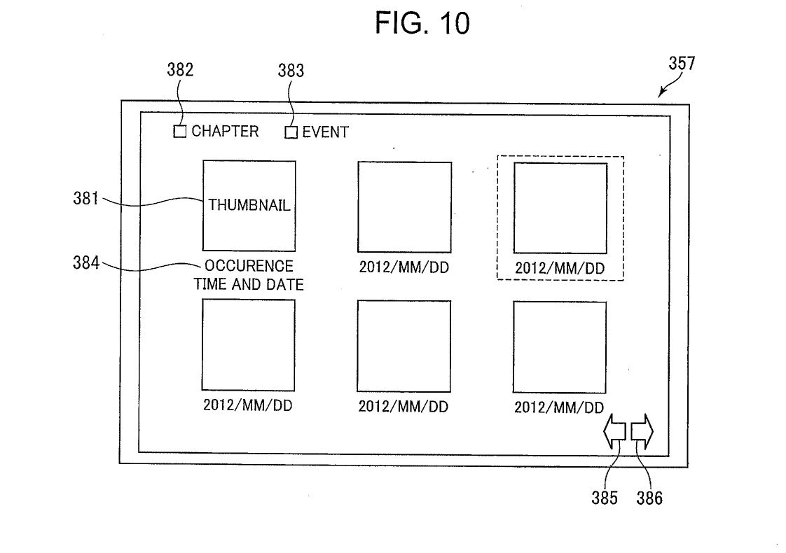

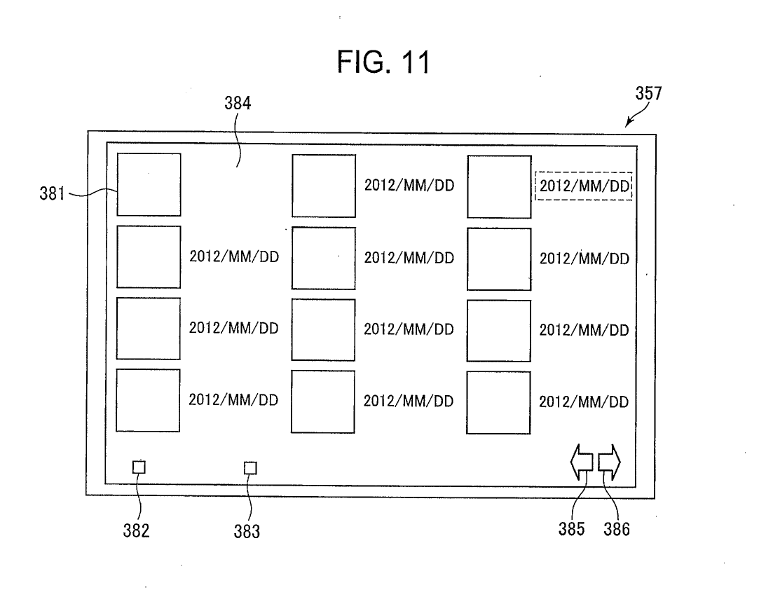

The video-playing process is described below with reference to FIGS. 9-11. FIG. 9 is a flowchart showing a video-playing process. FIGS. 10 and 11 show the monitor 351 that displays the search result. The video-playing process begins when a user gives an instruction for playing a video to the processor 300 by operating the user interface 352 and the touch panel 356.

In Step S81, a video file is retrieved from the recorder 353. In the next Step S82, it is determined whether or not search has been conducted. A search is determined to have been undertaken in the case that a user gives an instruction of search to the processor 300 by operating the user interface 352 and the touch panel 356. In the case that a search was carried out, the process proceeds to Step S83, otherwise, it proceeds to Step S91.

In the next Step S83, it is determined which event or chapter is searched for based on the search information that is input by a user. In the case that an event is searched for, the process proceeds to Step S84, otherwise it proceeds to Step S85.

In Step S84, a search is conducted for all of the events that are recorded in a video file. In Step S85, a search is conducted for all of the chapters in a video file.

In the next Step S86, the monitor 351 displays the search result. FIGS. 10 and 11 show a screen 357 of the monitor 351. The screen 357 displays the search result. With reference to FIG. 10, the screen 357 displays a thumbnail 381 of a frame image that corresponds to the search result, and occurrence time and date of an event. A chapter check box 382 that is used to select a chapter and an event check box 383 that is used to select an event are displayed on the upper left side of the screen 357. A rewind button 385 and a forward button 386 are displayed on the lower right side of the screen 357. FIG. 11 shows the search result displayed in another format. The screen 357 displays a thumbnail 381 of a frame image that corresponds to the search result, and occurrence time and date 384 of an event. The chapter check box 382 and the event check box 383 are displayed on the lower left side of the screen 357. The chapter check box 382 and the event check box 383 shown in FIGS. 10 and 11 are described hereinafter. When a user clicks the rewind button 385 with a mouse, etc., the screen 357 displays a frame image that is photographed before the currently displayed time. When a user clicks the forward button 386, the screen 357 displays a frame image that is photographed after the currently displayed time. The screen 357 may display the event ID, the chapter ID, occurrence time of event or chapter, the chapter number, name and occurrence time of event, patient information, information of observer, and time and date of observation, etc.

In Step S87, a user selects a search parameter from the search results displayed on the screen 357. That is, a user selects a search parameter from the event ID, chapter number, and occurrence time of event that are displayed on the screen 357. For example, when a user checks the chapter checkbox 382 with a mouse, the search target is determined to be a chapter, and when a user checks the event checkbox 383, the search target is determined to be a event.

In Step S88, a frame image that corresponds to the search parameter that is selected by a user in Step S87 is searched for from a table that is stored in the recorder 353.

In Step S89, it is determined whether or not the condition for playing a video has been preconfigured. The condition for playing a video is preconfigured according to an event and chapter selected by a user, and is, for example, playing from a frame image that corresponds to the search parameter, playing from a frame image that goes back a predetermined number of time periods before a frame image that corresponds to the search parameter, and the predetermined time period. In the case that the condition for playing a video is preconfigured, the process proceeds to Step S90, otherwise it proceeds to Step S91.

In Step S90, the frame image from which a video is played is determined. In Step S91, a video starts playing from the determined frame image in the case that the frame image is determined in Step S90. In the case that the frame image is not determined in Step S90, a video starts playing from a frame image that corresponds to the search parameter in Step S88.

In Step S92, it is determined whether a user stops playing a video or not. In the case that a user stops playing a video, the process proceeds to Step S93, otherwise, it returns to Step S91 and continues playing the video.

In Step S93, a video is stopped, and the process ends.

The process of Step S81 may be carried out right before Step S91. The processor 300 can search for an event and chapter in a plurality of video files that are stored in the recorder 353.

According to the embodiment, the location of a specific still image in a video is recorded, and the location of the specific still image is easily searched for.

A frame image that corresponds to an event has a high possibility that a user will pay attention to it. According to the embodiment, after an observation a user can easily search for a frame that caught his attention because the event is recorded in a video file. A frame image that a user paid attention to can be easily searched for because one observation can be stored as one file, which makes file management easier.

In a wait-and-see approach, an observation location can be easily found when a frame image is searched for with the name of patient, time and date of observation, and event.

A video is compressed, so that the entire observation process is recorded even in the case that the observation takes a long time. Therefore, a user will not lose any part of an observation.

A still image can be created from a recorded video, so that a user need not operate the still-image recording switch 222 during observation or a surgical procedure.

Sound and video are simultaneously recorded, so that all of the data produced during a surgical procedure is recorded.

A patient can watch a recorded video after a surgical procedure, so that the patient can understand precisely what occurred during the surgical procedure. A third party can study a surgical procedure with a recorded video.

Note that the imaging sensor is not limited to a CCD, and may be a solid-state image sensing device, e.g., a CMOS. The methods of compressing still images and video are not limited to those above.

Note that an event ID need not be preliminarily decided; it may be decided when an event occurs. An event ID and event number may be represented by a character, e.g., an alpha-numeric character that can be processed by a computer.

Note that a chapter ID need not be preliminarily decided; it may be decided when a chapter is occurs. A chapter ID and chapter number may be represented by a character, e.g., alpha-numeric character that can be processed by a computer.

In the video recording process, a video file may not be sent to the recorder 353 and may be sent to the server 390 instead. The file creating controller 326 may send a still image and a video to the server 390 through a wired LAN. In this case, the video file may not be sent over a wireless LAN, and may be sent through a peer-to-peer connection to the sever 390 instead.

Although the embodiment of the present invention has been described herein with reference to the accompanying drawings, obviously many modifications and changes may be made by those skilled in this art without departing from the scope of the invention.

The present disclosure relates to subject matter contained in Japanese Patent Application No. 2012-178421 (filed on Aug. 10, 2012), which is expressly incorporated herein, by reference, in its entirety.

1. An endoscope comprising:
a video compiler that continuously receives image signals and creates a video file with the image signals; and
a recorder that receives the video file and stores the video file;
the video compiler writing a flag into the video file when a first event occurs.

2. The endoscope according to claim 1, further comprising plurality of operators, wherein the video compiler writes a flag into the video file when the operator is operated.

3. The endoscope according to claim 2, wherein the flag has an event ID, and the video compiler assigns an event ID for each operation of the operators.

4. The endoscope according to claim 3, wherein the video compiler creates observation information that corresponds to the event ID, the recorder stores the event ID and the observation information so as to correlate with one another.

5. The endoscope according to claim 1, wherein the video compiler writes the flag into the video file for each time interval from the begging of recording of the video.

6. The endoscope according to claim 5, wherein the flag comprises a chapter ID.

7. The endoscope according to claim 6, wherein the video compiler creates observation information that corresponds to the chapter ID, and the recorder stores the chapter ID and the observation information so as to correlate with one another.

8. The endoscope according to claim 1, further comprising a searcher that searches for the flag stored in the video file.

9. The endoscope according to claim 4, further comprising a searcher that searches for the flag stored in the video file, and an inputter that receives search information, wherein the inputter retrieves observation information that corresponds to the received search information from the recorder, retrieves the event ID that corresponds to the retrieved observation information from the recorder, and finds an image that corresponds to the retrieved event ID in the video file.

10. The endoscope according to claim 7, further comprising a searcher that searches for the flag stored in the video file, and an inputter that receives search information, wherein the inputter retrieves observation information that corresponds to the received search information from the recorder, and retrieves the chapter ID that corresponds to the retrieved observation information from the recorder, and finds an image that corresponds to the retrieved chapter ID in the video file.
